(19) United States
(12) Patent Application Publication
(10) Pub. No.: US 2010/0267103 A1
(43) Pub. Date: Oct. 21, 2010

(54) FERMENTATION METHOD OF ANTLERS

(76) Inventors: Sin-Kyu Kim, Chungcheongnam-do (KR); Jin-Sung Kim, Seoul (KR)

Correspondence Address: Hershkovitz & Associates, LLC, 2845 Duke Street, Alexandria, VA, 22314, US

(21) Appl. No.: 12/745,890

(22) PCT Filed: May 19, 2008
(86) PCT No.: PCT/KR08/02784

§ 371 Date: Jun. 2, 2010

(30) Foreign Application Priority Data
Dec. 20, 2007 (KR) 10-2007-0133992

(51) Int. Cl. C12P 1/04; C12P 1/00

(52) U.S. Cl. 435/170; 435/41

(57) ABSTRACT

A method of fermenting animal horny substance, which includes a step of fermenting the mixture of animal horny substance and rice straw in order to ferment animal horny substance by using rice straw, so that not only water-soluble effective components in animal horny substance but also non-water-soluble effective components can be ingested.

### TECHNICAL FIELD

The present invention relates to a method of fermenting animal horny substance, and more particularly, to a method of fermenting animal horny substance, which includes a step of fermenting the mixture of animal horny substance and rice straw in order to ferment animal horny substance by using rice straw, so that not only water-soluble effective components in animal horny substance but also non-water-soluble effective components can be ingested.

### BACKGROUND ART

In general, according to oriental medicine, the deer antlers are an animal natural medicine known to be effective for physical strength reinforcement, recovery from the physical enervation after illness, neurasthenia, dizziness, anemia, lumbago, knee neuralgia, uterine bleeding, leucorrhea, sexual function reinforcement and spermatorrhea.

The deer antlers are collected from the horns, which have not become bony tissue, of male animals belonging to the family of deer, such as Cervus Nippon Temminck, Cervus elaphus L, Cervus uuicolor kerr and C. albirostris Przewalski, at least after 3 years of their birth.

According to the literatures of Oriental medicine, the dried deer antlers used as medicine have been known to contain 10% to 12% of water, 49% to 60% of organic substances and 7% to 12% of water-soluble substances, although there are some variations in the composition depending on where they are produced. They contain 0.3% to 2.3% of organic solvent-soluble substances, 26% to 41% of ash, phosphorous, magnesium and calcium; while the water-soluble substances include collcide and a very small amount of estron. Particularly the pantocrium isolated from deer antlers are known to have the functions of improving health, of pseudo-hormone and of diuresis.

Since the deer antlers are an animal Oriental medicine, usually they have been boiled to be taken, just like general Oriental herb medicine. Boiling, however, allows the absorption of only the substances eluted into water.

Of all the effective components of deer antlers, the water-soluble components eluted into water (small amount of estron and colloid) are only about 7% to 12%; thus the non-water-soluble effective components of deer antlers have not been extracted or isolated, but wasted.

Previously filed Korean Patent Application Publication No. 2001-0100177, which concerns a method of ingesting the effective components of deer antlers, disclosed the stamina food to which Oriental medicine was applied; and Korean Patent Application Publication No. 2001-0088465 disclosed a soup of deer antlers, which, however, also had the problem of the non-water-soluble effective components not being able to be ingested.

In addition, Korean Patent Application Publication No. 2004-0092951 and Korean Patent Application Publication No. 2002-0084360 disclosed the beverages in which deer antlers was used; and Korean Patent Application Publication No. 1998-0061431 and Korean Patent Application Publication No. 2000-0072546 disclosed the deer antlers tea and the deer antlers alcoholic beverage; Korean Patent Application Publication No. 2000-0074565 disclosed the deer antlers coffee; and Korean Patent Application Publication No. 2001-0008494 disclosed the deer antlers bean curd. These previously filed patents, however, again were successful only in extracting water-soluble components.

Korean Patent Application Publication No. 2002-14395 and Korean Patent Application Publication No. 10-2002-65197 disclosed a protein hydrolysis extraction method, which, however, had the problem of inefficient extraction of the non-water-soluble efficient substances. Korean Patent Application Publication 206181 used a bacterial strain of Py-92, which was isolated from animal intestines and which belongs to the genus Bacillus, to extract deer antlers; which, however, had a problem that the isolation of the strain was too difficult and inconvenient to be practiced.

Korean Patent No. 75600 disclosed a method of extraction using organic solvent, and Korean Patent No. 94501 disclosed a pressurized extraction method; which, however, had a problem that the yield of the extracted effective components was poor. Korean Patent No. 51180 disclosed a method of mechanical grinding of deer antlers; which, however, had a problem that the deer antlers ground in such a way is difficult to be ingested and absorbed.

### DISCLOSURE OF INVENTION

### Technical Problem

The present invention has been made to solve the foregoing problems with the prior art, and therefore the present invention is directed to a method of fermenting deer antlers, which is an animal horny substance, using rice straw, so that not only the water-soluble effective components but also the non-water-soluble effective components contained in the animal horny substance can be ingested.

The present invention is also directed to make deer antlers be used in easy and diversified ways, by fermenting deer antlers, which is an animal horny substance, using the enzymes contained in rice straw, thereby making deer antlers into low molecular weight forms so that it can be taken as powder and be easily digested and absorbed. The present invention is further directed to preserve the heat-labile effective components by not using the hot water extraction method.

The present invention is further directed to provide easy methods of handling and maintaining horny substance, by making it into powder and preventing the feeling of disgust caused by the order when handling it.

### Technical Solution

According to an aspect of the present invention, there is provided a method of fermenting animal horny substance. The method includes a step of fermenting a mixture of animal horny substance and rice straw.

Further, the animal horny substance can be a mixture of at least one kind selected from the group consisting of deer antlers and cow horns.

The method may further include steps of:

prior to the fermentation of the animal horny substance, putting the animal horny substance in 3% to 5% alcohol for 10 hours to 12 hours before the fermentation;

washing with purified water and drying the horny substance that has been put in the alcohol;

heating the dried horny substance in purified water at 85° C. to 95° C. for 3 hours to 6 hours; and

cooling the heated horny substance to 30° C. to 40° C.

### Advantageous Effects

As set forth above, the present invention uses rice straw to ferment animal horny substance such as deer antlers, to provide the effects of allowing the ingestion of not only the water-soluble effective components but also the non-water-soluble effective components contained in the animal horny substance; the easy digestion and absorption by making deer antlers into low molecular weight forms using bacterial strain; the ease of use by making them into powder; and the preservation of the heat-labile effective components by not using a hot water extraction method.

Furthermore, the powdered horny substance has the effect of allowing the ease of use for handling and maintenance, without decomposition even in the hot and humid summer, and also without the order that causes the feeling of disgust.

### BEST MODE FOR CARRYING OUT THE INVENTION

The present invention will be described in detail hereinafter.

The invention involves the fermentation of the mixture of animal horny substance and rice straw, wherein the rice straw is used as the provider of the strain of Bacillus.

The rice straw can be obtained sufficiently from farmhouses; the waste straws which contain about 10% to 20% of water are used as they are, after washing them clean, without other processes. There is therefore no need of isolating the strain of Bacillus from the rice straw, which makes the method be conveniently practiced, while providing sufficient ability to decompose the proteins at lower costs.

The animal horny substance may be the mixture of at least one kind selected from the group consisting of deer antlers, deer horn and cow horns, but is not limited such mixtures.

The fermentation of the mixture of animal horny substance and rice straw makes the horny substance soft; then the softened substance can be processed into fine light brown powder with the yield of higher than 90%, which allows the sufficient ingestion of the water-soluble effective substances as well as the non-water-soluble substances, with better efficacy and reduced costs.

Further, the substance fermented using the rice straw is made into low molecular weight forms, allowing not only easy digestion and absorption but also easy handling.

Hereunder the present invention will be described in more detail.

The animal horny substance may be pre-treated as follows in order to make the fermentation even easer:

The animal horny substance is first put in alcohol (3% to 5%) for 10 hours to 12 hours. This is to remove a lot of impurities and parasites from the skin of the animal horny substance. If the alcohol content is below 3% by weight or the fermentation time is less than 10 hours, there is little effect; while if the alcohol content is above 5% or the fermentation time is more than 12 hours, some amounts of the effective components contained in the animal horny substance may be eluted.

The horny substance that have been put in the alcohol is washed with purified water and is dried, and 4 times to 5 times (by volume) of purified water is added to the dried horny substance, which is then heated at 85° C. to 95° C. for 3 hours to 6 hours to make the horny substance soft. The temperature is set so in consideration of the boiling point of the purified water, and the heating time to obtain the best softness of the horny substance.

The volume of the purified water reduces to ½ of the original volume when the heating have been finished; and the heated horny substance is cooled down to 30° C. to 40° C. to complete the pre-treatment.

Upon completion of the pre-treatment, the pre-treated horny substance is fermented with rice straw. The heated horny substance is fermented as it is in the solution, and the purpose of cooling the heated horny substance to 30° C. to 40° C. is to make the following process easy, which is fermentation.

For the fermentation, the rice straws are layered (about 1 cm thick) at the bottom of a sterilized fermentor, on which about the half amount of the heated horny substance (including the solution) is added, on which again the rice straws were layered (about 1 cm thick), on which then the remaining half is added, on which again the rice straws is layered (about 1 cm thick). A sterilized wooden panel or a ceramic bowl is put on the top layer to press down the rice straws, which is then subjected to fermentation at 40° C. to 55° C. for 5 days to 7 days.

The amount of the rice straws or the amount of the aliquoted horny substance depends on the amount of the whole horny substance, and there is no limitation to the amount. The reason for maintaining the temperature between 40° C. and 55° C. is that fermentation is not easy below 40° C., and that, above 55° C., overheating occurs during the fermentation. In addition, if the fermentation time is less than 5 days, it is not fermented sufficiently; while if it is more than 7 days, it becomes rotting. Thus 5 days to 7 days of fermentation is appropriate.

Upon the completion of the fermentation, alcohol is added to the fermented animal horny substance, which is evaporation concentrated (bp₂₀, 50° C. to 60° C.) to a solution of 60 brix to 70 brix, which is then hot air dried or freeze dried to be made into powder. The powder form makes easy not only the ingestion of animal horny substance but also the handling and maintenance. The methods of concentrating or drying are not limited to the described ones, and of course other methods may be used.

The powdered horny substance must be autoclaved (0.7 Psi to 1.3 Psi, 5 min. to 15 min.) to get rid of any bacterial strain, which is then vacuum-packaged to make distribution and maintenance even easier.

In addition to the described pre-treatment methods, particularly the cervi cornu may be put in an organic acid solution with a pH of 2.0 to 4.0 for 5 hours to 10 hours and then be washed before the fermentation of the horny substance using rice straw as described. The treatment with the acidic solution with a pH of 2.0˜4.0 is for the cases where a small amount of proteins present in the animal horny substance may make the softening difficult. If pH is too low, calcium is eluted; while the pre-treatment with the organic acid before the fermentation with rice straw facilitates the fermentation.

The method of fermenting animal horny substance according to the present invention as described above allows the ingestion of 100% of the animal horny substance, thereby providing excellent efficacy; a less amount of the animal horny substance of the present invention can provide the same efficacy as was provided by a more amount of it prepared by previous methods. The fermentation causes deformation of molecules from high molecular weight to low molecular weight, which allows excellent absorption and better digestion. Further, the powdered form not only makes maintenance and distribution easy, but it is also convenient to take and there is no worry of decomposition during maintenance. In addition, the powdered form can be used as raw materials for cosmetics, and also as medicines and foods.

The present invention will be described in more detail with respect to Example hereunder.

### EXAMPLE

The fur and impurities were removed from the skin of 1 kg of dried deer antlers, which was put in alcohol (3% to 5%) for 10 hours to 12 hours, was washed with purified water more than 3 times, was dried in the sun light for 3 hours to 4 hours; and then was finely sliced after all the water was evaporated from the surface thereof.

51 to 71 of purified water was added to the 1 kg of the finely sliced deer antlers in a stainless steel container, which was heated at 85° C. to 95° C. for 4 hours to 6 hours until the added water was reduced to half the original volume, and was then cooled to 30° C. to 40° C.

The cleansed rice straws (10% to 15% of water content) were layered (50 g in a 1 cm thick layer) at the bottom of a specially manufactured stainless steel fermentor container, on which about a half amount of the deer antlers was added, on which again 20 g of the rice straws were layered, on which the remaining deer antlers were added with a solution, on which another layer of 30 g of rice straws was carefully covered, on which a stone or ceramic bowl was put to press the contents down. Then, the container was covered with a lid, and the contents were subjected to fermentation at 40° C. to 50° C. for 5 days to 7 days.

Upon the completion of the fermentation, all the deer antlers were liquefied and mixed with the added water. After isolating and removing the added rice straws, 0.51 to 11 of alcohol (5% to 6%) was added, followed by refluxing for 1 hour to 1.5 hours to get rid of odors. The resultant contents were then evaporation concentrated (bp20, 50° C. to 60° C.) to 60 brix to 70 brix.

The concentrate was freeze dried or hot air dried into powder, which was essentially autoclaved (0.7 Psi to 1.3 Psi, 5 min to 15 min); the deer antler powder was vacuum packaged to be ready as a product.

The present invention was described with respect to the example above, but the invention is not limited to the example, and of course a variety of modified examples are possible within the scope and idea of the invention.

1. A method of fermenting animal horny substance, comprising:
fermenting a mixture of animal horny substance and rice straw.

2. The method according to claim 1, wherein the animal horny substance is a mixture of at least one kind selected from the group consisting of deer antlers, cervi cornu and cow horns.

3. The method according to claim 1, further comprising:
prior to the fermentation of the animal horny substance, putting the animal horny substance in 3% to 5% alcohol for 10 hours to 12 hours;
washing with purified water and drying the animal horny substance that has been put in the alcohol;
heating the dried horny substance in purified water at 85° C. to 95° C. for 3 hours to 6 hours; and
cooling the heated horny substance to 30° C. to 40° C.

4. The method according to claim 1, wherein the fermentation is carried out at 40° C. to 55° C. for 5 days to 7 days.

5. The method according to claim 1, further comprising:
prior to the fermentation of the animal horny substance, putting the animal horny substance in an acidic solution with a pH of 2.0 to 4.0 for 5 hours to 10 hours, followed by washing.

6. The method according to claim 1, wherein the rice straw comprises
a strain of Bacillus.

7. The method according to claim 1, further comprising:
after the fermentation, evaporation-concentrating the fermented animal horny substance;
powdering the evaporation-concentrate; and
autoclaving the powdered horny substance.

8. The method according to claim 2, further comprising:
prior to the fermentation of the animal horny substance, putting the animal horny substance in 3% to 5% alcohol for 10 hours to 12 hours;
washing with purified water and drying the animal horny substance that has been put in the alcohol;
heating the dried horny substance in purified water at 85° C. to 95° C. for 3 hours to 6 hours; and
cooling the heated horny substance to 30° C. to 40° C.

9. The method according to claim 2, wherein the fermentation is carried out at 40° C. to 55° C. for 5 days to 7 days.

10. The method according to claim 2, further comprising:
prior to the fermentation of the animal horny substance, putting the animal horny substance in an acidic solution with a pH of 2.0 to 4.0 for 5 hours to 10 hours, followed by washing.

11. The method according to claim 2, wherein the rice straw comprises
a strain of Bacillus.

12. The method according to claim 2, further comprising:
after the fermentation, evaporation-concentrating the fermented animal horny substance;
powdering the evaporation-concentrate; and
autoclaving the powdered horny substance.
